(19) United States
(12) Patent Application Publication
(10) Pub. No.: US 2025/0367456 A1
(43) Pub. Date: Dec. 4, 2025

(54) REMOTE CRT PARAMETER CHANGE RESPONSE EVALUATION AND PROGRAMMING

(71) Applicant: Cardiac Pacemakers, Inc., St. Paul, MN (US)

(72) Inventors: Sean Thomas Horan, Maplewood, MN (US); Wyatt Keith Stahl, Little Canada, MN (US); Robert D. Brock, II, South Saint Paul, MN (US); Kevin G. Wika, Blaine, MN (US)

(21) Appl. No.: 19/220,201

(22) Filed: May 28, 2025

Related U.S. Application Data
(60) Provisional application No. 63/655,782, filed on Jun. 4, 2024.

(51) Int. Cl. A61N 1/37; A61N 1/362; A61N 1/372

(52) U.S. Cl. CPC A61N 1/3702; A61N 1/362; A61N 1/37258; A61N 1/37264

(57) ABSTRACT

Systems and methods are disclosed to evaluate patient response to cardiac rhythm management therapy and remotely reprogram an implantable medical device, including receiving physiologic information of a patient in a first time period responsive to a first cardiac rhythm management therapy, determining a first patient response metric indicative of patient response to the first cardiac rhythm management therapy, generating a reprogramming recommendation for the implantable medical device including a second cardiac rhythm management therapy based on the first patient response metric, and remotely reprogramming the implantable medical device to provide the second cardiac rhythm management therapy to the patient in a second time period.

### CLAIM OF PRIORITY

This application claims the benefit of U.S. Provisional Application No. 63/655,782, filed on Jun. 4, 2024, which is hereby incorporated by reference in its entirety.

### TECHNICAL FIELD

This document relates generally to medical devices and more particularly to remote cardiac resynchronization therapy parameter change response evaluation and programming.

### BACKGROUND

Heart failure (HF) is a reduction in the ability of the heart to deliver enough blood to meet bodily needs. Heart failure patients commonly have enlarged hearts with weakened cardiac muscles, resulting in reduced contractility and poor cardiac output. Signs of heart failure include pulmonary congestion, edema, difficulty breathing, etc. Heart failure is often a chronic condition, but can also occur suddenly, affecting the left, right, or both sides of the heart. Causes of heart failure include coronary artery disease, myocardial infarction, high blood pressure, atrial fibrillation, valvular heart disease, alcoholism, infection, cardiomyopathy, or one or more other conditions leading to a decreased pumping efficiency of the heart.

Ambulatory medical devices, including implantable, subcutaneous, wearable, or one or more other medical devices, etc., can monitor, detect, or treat various conditions including heart failure, atrial fibrillation, etc. Ambulatory medical devices can include sensors to sense physiologic information from a patient and one or more circuits to detect one or more physiologic events using the sensed physiologic information or transmit sensed physiologic information or detected physiologic events to one or more remote devices. Additionally, ambulatory medical devices can be configured to provide electrical stimulation or one or more other therapies or treatments to the patient, such as to improve cardiac function, etc.

Ambulatory patient monitoring can provide early detection of worsening patient condition, including worsening heart failure or atrial fibrillation. Accurate identification of patients or groups of patients at an elevated risk of future adverse events may control mode or feature selection or resource management of one or more medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of medical devices, and reduce healthcare costs. In addition, in situations where different operating modes, features, or therapies are available, correctly monitoring, detecting, and identifying patient status, including improving or worsening patient condition, and modifying one or more medical device functions based thereon, can improve medical device efficiency, such as by reducing unnecessary resource consumption, thereby extending the usable life of the ambulatory medical device.

### SUMMARY

Systems and methods are disclosed to evaluate patient response to cardiac rhythm management therapy and remotely reprogram an implantable medical device, including receiving physiologic information of a patient in a first time period responsive to a first cardiac rhythm management therapy, determining a first patient response metric indicative of patient response to the first cardiac rhythm management therapy, generating a reprogramming recommendation for the implantable medical device including a second cardiac rhythm management therapy based on the first patient response metric, and remotely reprogramming the implantable medical device to provide the second cardiac rhythm management therapy to the patient in a second time period.

An example of subject matter (e.g., a computing device or a system for evaluating patient response to cardiac rhythm management therapy by an implantable medical device and for remotely reprogramming the implantable medical device) may comprise means for receiving physiologic information of a patient from the implantable medical device, including physiologic information of the patient in a first time period responsive to the implantable medical device providing a first cardiac rhythm management therapy to the patient, means for determining, using the physiologic information of the patient in the first time period, a first patient response metric indicative of patient response to the first cardiac rhythm management therapy, means for generating, based on determining that a value of the first patient response metric exceeds a first threshold, a reprogramming recommendation for the implantable medical device including a second cardiac rhythm management therapy based on the first patient response metric, and means for remotely reprogramming the implantable medical device to provide a second cardiac rhythm management therapy to the patient in a second time period subsequent to the first time period, wherein the first time period includes a post-implant time period following implant of the implantable medical device in the patient.

An example of subject matter (e.g., a computing device or a system for evaluating patient response to cardiac rhythm management therapy by an implantable medical device and for remotely reprogramming the implantable medical device), which may be combined with any one or more examples described herein, may comprise one or more processors and one or more memory devices storing instructions, which when executed by the one or more processor, cause the one or more processors to perform operations comprising receiving physiologic information of a patient from the implantable medical device, including physiologic information of the patient in a first time period responsive to the implantable medical device providing a first cardiac rhythm management therapy to the patient, determining, using the physiologic information of the patient in the first time period, a first patient response metric indicative of patient response to the first cardiac rhythm management therapy, generating, based on determining that a value of the first patient response metric exceeds a first threshold, a reprogramming recommendation for the implantable medical device including a second cardiac rhythm management therapy based on the first patient response metric, and remotely reprogramming the implantable medical device to provide a second cardiac rhythm management therapy to the patient in a second time period subsequent to the first time period, wherein the first time period includes a post-implant time period following implant of the implantable medical device in the patient.

In an example, which may be combined with any one or more examples described herein, the operations may further comprise receiving an indication of a time of implant of the implantable medical device in the patient, wherein the post-implant time period comprises a pre-determined time period from the time of implant of the implantable medical device.

In an example, which may be combined with any one or more examples described herein, the pre-determined time period includes a time period of 2 to 4 weeks after the time of implant of the implantable medical device or after a recovery period after the time of implant of the implantable medical device.

In an example, which may be combined with any one or more examples described herein, the operations may further comprise receiving physiologic information of the patient from the implantable medical device, including physiologic information of the patient in the second time period responsive to the implantable medical device providing the second cardiac rhythm management therapy to the patient, determining, using the physiologic information of the patient in the second time period, a second patient response metric indicative of patient response to the second cardiac rhythm management therapy, generating, based on determining that a value of the second patient response metric exceeds a second threshold, a reprogramming recommendation for the implantable medical device including a third cardiac rhythm management therapy based on the second patient response metric, and remotely reprogramming the implantable medical device to provide a third cardiac rhythm management therapy to the patient in a third time period subsequent to the second time period.

In an example, which may be combined with any one or more examples described herein, determining the second patient response metric includes updating the first patient response metric.

In an example, which may be combined with any one or more examples described herein, generating the reprogramming recommendation including the third cardiac rhythm management therapy comprises reverting from the second cardiac rhythm management therapy to the first cardiac rhythm management therapy if the second patient response metric indicates a worse patient status than the first patient response metric.

In an example, which may be combined with any one or more examples described herein, the operations may further comprise generating, based on determining that the value of the first patient response metric exceeds the first threshold, an alert to a user or process, and providing an indication of the value of the first patient response metric to the user or process.

In an example, which may be combined with any one or more examples described herein, the operations may further comprise scheduling an in-clinic follow-up appointment or adjusting a follow-up schedule for the patient based on the first patient response metric.

In an example, which may be combined with any one or more examples described herein, the first cardiac rhythm management therapy includes a first mode or a first set of therapy parameters and the second cardiac rhythm management therapy includes a second mode different than the first mode or a second set of therapy parameters different than the first set of therapy parameters.

In an example, which may be combined with any one or more examples described herein, the first mode comprises a CRT therapy mode and the second mode comprise an MSP mode different than the CRT therapy mode.

An example of subject matter (e.g., a method for evaluating patient response to cardiac rhythm management therapy by an implantable medical device and for remotely reprogramming the implantable medical device) may comprise receiving physiologic information of a patient from the implantable medical device, including physiologic information of the patient in a first time period responsive to the implantable medical device providing a first cardiac rhythm management therapy to the patient, determining, using the physiologic information of the patient in the first time period, a first patient response metric indicative of patient response to the first cardiac rhythm management therapy, generating, based on determining that a value of the first patient response metric exceeds a first threshold, a reprogramming recommendation for the implantable medical device including a second cardiac rhythm management therapy based on the first patient response metric, and remotely reprogramming the implantable medical device to provide a second cardiac rhythm management therapy to the patient in a second time period subsequent to the first time period, wherein the first time period includes a post-implant time period following implant of the implantable medical device in the patient.

In an example, which may be combined with any one or more examples described herein, receiving an indication of a time of implant of the implantable medical device in the patient, wherein the post-implant time period comprises a pre-determined time period from the time of implant of the implantable medical device.

In an example, which may be combined with any one or more examples described herein, the pre-determined time period includes a time period of 2 to 4 weeks after the time of implant of the implantable medical device or after a recovery period after the time of implant of the implantable medical device.

In an example, which may be combined with any one or more examples described herein, the subject matter may optionally comprise receiving physiologic information of the patient from the implantable medical device, including physiologic information of the patient in the second time period responsive to the implantable medical device providing the second cardiac rhythm management therapy to the patient, determining, using the physiologic information of the patient in the second time period, a second patient response metric indicative of patient response to the second cardiac rhythm management therapy, generating, based on determining that a value of the second patient response metric exceeds a second threshold, a reprogramming recommendation for the implantable medical device including a third cardiac rhythm management therapy based on the second patient response metric, and remotely reprogramming the implantable medical device to provide a third cardiac rhythm management therapy to the patient in a third time period subsequent to the second time period.

In an example, which may be combined with any one or more examples described herein, determining the second patient response metric includes updating the first patient response metric.

In an example, which may be combined with any one or more examples described herein, generating the reprogramming recommendation including the third cardiac rhythm management therapy comprises reverting from the second cardiac rhythm management therapy to the first cardiac rhythm management therapy if the second patient response metric indicates a worse patient status than the first patient response metric.

In an example, which may be combined with any one or more examples described herein, the subject matter may optionally comprise generating, based on determining that the value of the first patient response metric exceeds the first threshold, an alert to a user or process, and providing an indication of the value of the first patient response metric to the user or process.

In an example, which may be combined with any one or more examples described herein, the subject matter may optionally comprise scheduling an in-clinic follow-up appointment or adjusting a follow-up schedule for the patient based on the first patient response metric.

In an example, which may be combined with any one or more examples described herein, the first cardiac rhythm management therapy includes a first mode or a first set of therapy parameters and the second cardiac rhythm management therapy includes a second mode different than the first mode or a second set of therapy parameters different than the first set of therapy parameters.

In an example, which may be combined with any one or more examples described herein, the first mode comprises a CRT therapy mode and the second mode comprise an MSP mode different than the CRT therapy mode.

In an example, a system or apparatus may optionally combine any portion or combination of any portion of any one or more of the examples described herein, may optionally combine any portion or combination of any portion of any one or more of the examples described herein to comprise “means for” performing any portion of any one or more of the functions or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates example schedules for implant and follow-up for an ambulatory medical device.

FIG. 2 illustrates a table of example parameter settings.

FIG. 3 illustrates a diagram of an example machine learning model.

FIG. 4 illustrates an example method.

FIG. 5 illustrates an example medical device system.

FIG. 6 illustrates an example patient management system and portions of an environment in which the system may operate.

FIG. 7 illustrates an example implantable medical device (IMD) electrically coupled to a heart.

FIG. 8 illustrates an example machine upon which any one or more of the techniques discussed herein may perform.

### DETAILED DESCRIPTION

Ambulatory medical devices can be implanted in or otherwise positioned on or about patients to monitor physiologic information, such as cardiac electrical, heart sound, respiration (e.g., respiration rate (RR), tidal volume (TV), rapid shallow breathing index (RSBI), etc.), impedance (e.g., intrathoracic impedance (ITTI)), pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include one or more implantable or external (e.g., wearable) cardiac rhythm management devices configured to monitor or provide stimulation to the patient. Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICDs), subcutaneous implantable defibrillators (S-ICDs), cardiac resynchronization therapy defibrillators (CRT-Ds), insertable cardiac monitors (ICMs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (LV) (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency, such as by effectuating 100% cardiac capture from pacing stimulation, where cardiac capture can refer to LV cardiac capture, RV cardiac capture, or combinations thereof. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, HIS-bundle pacing, septal pacing, etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Medical devices, such as cardiac rhythm management devices, can provide different therapies using different therapy modes, however, with different power and resource requirements and varying effectiveness for different patients. A variety of therapy modalities are available to patients, but not all patients receive the optimal medical device, therapy mode, or therapy parameter settings at first programming. Recent literature suggests that nearly 40% of current pacing therapy is suboptimal, where suboptimal is defined as cardiac resynchronization therapy resulting in cardiac capture of one or more chambers (typically the LV for CRT patients generally) in less than 98% of cardiac beats. One common reason for suboptimal cardiac resynchronization therapy is inappropriate programming of parameter settings of medical devices (e.g., implantable medical devices) configured to provide cardiac resynchronization therapy (e.g., CRT devices).

When receiving a new medical device, patients may need to try several sets of parameter settings to receive sufficient or optimal therapy. In addition, in-clinic follow-up appointments are currently required as patient condition can change or response to existing devices or therapy can change over time, requiring additional changes to parameter settings that at one time were sufficient or optimal. In typical operation, a medical device, such as a cardiac resynchronization therapy device, is first programmed at a time of implant to a first operating mode, such as with respect to a first set of parameter settings, and then adjusted during scheduled in-clinic follow-up procedures with a clinician. Initial follow-up after implant (e.g., post-implant) or programming changes is generally a first time period, such as 4-6 weeks to allow patient recovery from the implant procedure and determination of baseline measurements for the patient from which to base future operation and monitoring, as well as to compare patient condition after implant or programming changes to the patient condition pre-implant or previous to programming changes. Subsequent follow-up after the initial follow-up can be less frequent, occurring, for example, every 3-6 months (e.g., at 6 months post-implant, at 12-months post-implant, etc.), or more or less as needed (e.g., between 1 and 12 months, etc.) depending on programming changes or changes in patient status or condition (e.g., a patient response metric). However, traditional follow-up appointments are in-person in a clinical setting and require travel for the patient, often at a substantial burden. In addition, programming changes may require additional follow-up, such as a new initial follow-up appointment and observation time, substantially increasing resources associated with programming the medical device and reducing the usable lifespan of the medical device during which the device is using limited resources to provide sufficient or optimal therapy.

The present inventors have recognized, among other things, systems and methods to determine which of multiple therapies may be more effective for a respective patient using physiologic information from the respective patient, without requiring in-person appointments, including implementing parameter setting changes (e.g., including changes in modes) from a medical device system including a remote component separate from the ambulatory medical device. In an example, instead of each change in parameter settings requiring an in-person clinic visit or follow-up appointment, the present inventors have recognized systems and methods to remotely monitor a physiologic status of the patient through the ambulatory medical device and determine patient status (e.g., a patient response metric) in response to an implemented set of parameter settings, such as a series of different sets of parameter settings received or authorized at a time of implant or initial programming.

For example, the systems and methods can be configured to determine a value or trend indicative of patient status in response to a first set of parameter settings (e.g., determining if the first set of parameter settings are beneficial to the patient or if a change in parameter settings are needed). If the determined patient status to the first set of parameter settings is below a threshold (e.g., an expected patient status or a relative threshold determined as a function of a previous patient status, etc.), a second set of parameter settings can be implemented remotely by the medical device system through the ambulatory medical device. Like with respect to the first set of parameter settings, the systems and methods can be configured to determine patient status in response to updated or changed parameter settings (e.g., the second set of parameter settings, etc.) to determine if the updated or changed parameter settings are beneficial to the patient or if additional changes in parameter settings or additional follow-up is needed. In certain examples, the determined patient status can be used to trigger an alert to a clinician or an adjustment to a follow-up schedule, such as if a value of the determined patient status is above or below one or more thresholds associated with an alert or follow-up requirement, etc.

As therapy progresses, physiologic characteristics of the heart change. Accordingly, even after an initial therapy has been optimized, it is important to periodically reassess and re-optimize therapy and parameter settings at extended intervals (e.g., yearly, etc.). Accordingly, even if patient status is above a healthy threshold, one or more parameter settings or modes can be adjusted to determine positive or negative patient response to the adjustments, such as to guide additional programming changes to continue to provide optimal therapy or parameter settings.

The present inventors have further recognized, among other things, an automated “operation system test” or “system self-evaluation” on device function or parameter settings to confirm continued optimal operation. In an example, one or more parameter settings can be changed or adjusted and one or more indications of patient condition (e.g., patient response metrics, indications of patient status, risk indications, etc.) can be determined in response thereto. In an example, if the indication of patient condition falls or deteriorates in response to the change or adjustment, device function or parameter settings can be programmed to revert to the previous operation. In other examples, one or more other changes or adjustments can be programmed, in contrast to such changes that provided the previous deterioration in patient condition, and a responsive indication of patient condition can be determined to guide additional changes, adjustments, or reversion to previous operation.

In certain examples, a clinician or a user can implement a stop action to revert or rollback a change in parameter settings in response to negative changes in patient response metrics (e.g., rates, thresholds, impedance, adverse medical events, etc.) after a change. Such stop action can be received by one or more circuits, for example, through an external or a remote device, and can include an open-ended stop action to revert to a previous operation or a time-based stop action allowing a user to provide a stop action to be implemented for a specific post-change time period (e.g., several hours, up to a day, etc.). In other examples, patient response metrics after a change in parameter settings can be evaluated by an external or remote device to recommend or implement a stop action or provide one or more alerts, etc., in response to negative or adverse findings.

Advantages of the systems and methods described herein include, among other things, optimized power usage by the ambulatory medical device, extending a usable lifespan of the limited power resources (e.g., battery) by more quickly optimizing therapy through successive changes in parameter settings, additionally include optimization of clinician resources and follow-up scheduling, as well as reducing the need for patient self-reporting of status following a change in parameter settings, allowing a more responsive and objective reporting and management system.

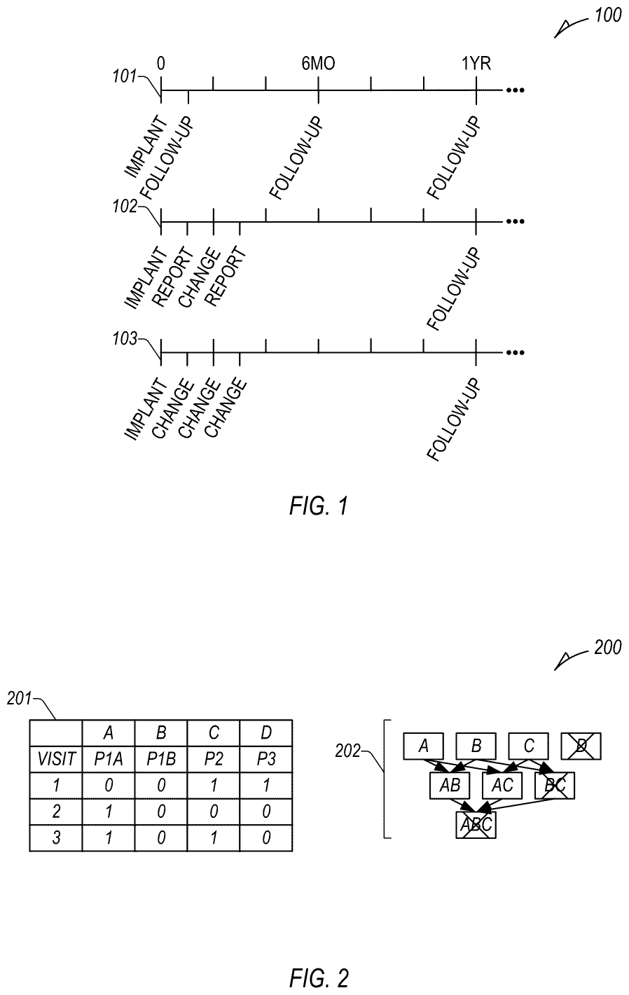

FIG. 1 illustrates example schedules 100 for implant and follow-up for an ambulatory medical device from time (0) at a time of implant through 1 year for three (3) scenarios including a first schedule 101 having an implant at time (0) with an initial follow-up (in-clinic) at 1 month (e.g., 4 weeks) after implant and subsequent follow-ups (in-clinic) at 6 months and 1 year. In this example, follow-up can include in-clinic follow-up. Subsequent follow-up can continue after 1 year at this or other durations. In an example, in the first schedule, changes to parameter settings can be determined and programmed to the ambulatory medical device by a clinician during follow-up appointments. In certain examples, similar to the initial follow-up at 1 month after implant, additional initial follow-up can be scheduled following each change in parameter settings.

A second schedule 102 illustrates an implant at time (0) with a report determined, such as by a patient management system, and provided to a clinician at a first time period (e.g., including a recovery period and time to determine a baseline response) after implant. In this example, the report can be provided remotely, without an in-clinic follow-up appointment. In response to the report, the clinician can program changes to the parameter settings, such as through the patient management system, to be implemented by the ambulatory medical device without an in-clinic follow-up. After a second time period (e.g., a subsequent recovery period) after the change in parameter settings, an additional report can be determined and provided to the clinician. In an example, the report can include a determined patient response metric or patient status or condition based upon received physiologic information for the respective time period, or a comparison of the determined patient response metric for the respective time period in contrast to a determined patient response metric for a previous time period, such as a time before implant, or a time before a prior change, etc. In an example, if no subsequent changes are provided or desired by the clinician, traditional in-clinic follow-up can continue 1 year or one or more other time periods or durations. Also, as noted above, it can be important to re-optimize at longer time periods (e.g., such as at 1 year or greater). In certain examples, it can be beneficial to perform re-optimization in the months or weeks prior to a scheduled in-clinic follow-up, such that the changes are before but close in time to a planned in-clinic visit, reducing subsequent follow-up appointments.

A third schedule 103 illustrates an implant at time (0) with changes automatically determined automatically by a patient management system, such as through one or more automated systems configured to analyze parameter settings and generate reprogramming recommendations for ambulatory medical devices to optimize or improve patient condition, such as by optimizing parameter settings to improve a percentage of confirmed cardiac capture resulting from pacing stimulation, etc. In certain examples, a clinician, at the time of implant or after a recovery period, can authorize a range of settings or a series of settings for the device to cycle and optimize through successive changes and analysis, remotely, without in-clinic requirements for each change in parameter settings. If responses are within acceptable guidelines or normal expected values, reports can be placed in the patient medical records without an alert to the clinician until the range or series of settings have been implemented and evaluated, or until a determined patient response exceeds a threshold.

In an example, the third schedule 103 illustrates three changes. In an example, the first period after implant at time (0) before the first change can include a recovery or monitoring period, or in other examples, a first therapy. Accordingly, in one example, the first change can include a change from the recovery or monitoring period to the first therapy, or a change from the first therapy to a second therapy. The second change (e.g., at 2 months) can include a subsequent change different than the first change. The third change (e.g., at 3 months) can include a subsequent change different than the first or second changes, or in certain examples, if one of the previous therapy or sets of parameter settings provided a better patient response metric (higher if a higher value of the patient response metric indicates a positive patient condition, lower if a higher value of the patient response metric indicates a worsening patient condition), the third change can include reverting back to the therapy having the better patient response metric (e.g., from a second therapy to a first therapy, etc.), and away from the therapy or time period indicating a worse patient status.

Although shown herein as specific time periods, such as pre-determined time periods, etc., in other examples, the rate of change, follow-up, report, recovery, or implementation of initial or subsequent programming or therapy can include one or more other time periods determined by a clinician, determined by patient response metrics or physiologic information of the patient, or one or more other time periods. In an example, a lock-out period can be implemented by one or more devices or circuits or set by a clinician to prohibit device changes in modes or parameter settings in the lock-out period (e.g., one week, two weeks, etc.) prior to a scheduled follow-up appointment, such as to avoid a changing patient condition during subsequent follow-up which may lead to additional unnecessary device changes, enforcing a lock-out period on the device side. In other examples, a follow-up schedule can be modified based on a time of most recent change in parameter settings, such as to avoid a changing patient condition during subsequent follow-up which may lead to additional unnecessary device changes, enforcing the lock-out period on the clinician side.

In an example, the remote patent management system can be configured to analyze different pacing parameters using artificial intelligence or machine learning, based on received physiologic information or separate therefrom, to identify optimal and suboptimal combinations of pacing parameters corresponding to one or more conditions, such as to optimize patient condition, to improve rates of cardiac capture resulting from pacing stimulation, to eliminate or reduce periods of suboptimal, missed, or reduced pacing, etc. Data can be collected and organized for analysis and identification of patterns. Models can be created based on the identified patterns, validated (e.g., using a percentage of confirmed LV cardiac capture, etc.), stored, and deployed. Additionally, deployed models can be monitored and updated as additional data is collected, including retraining as needed.

Medical device systems frequently analyze physiologic information between patients or with respect to one or more clinical thresholds to determine patient condition and optimize device settings. In other examples, analysis can focus on differences between the parameter settings themselves (e.g., without respect to patient physiologic information, determined indications of cardiac capture or reduced pacing, patient demographics, patient history, etc.), such that a determined similarity between different parameter settings for the same or different patients can be analyzed to identify sub-optimal settings or combinations of settings that may result in suboptimal, missed, or reduced pacing. In certain examples, parameter settings can be additionally analyzed with respect to one or more of patient physiologic information (e.g., to identify similar patients, etc.), determined indications of cardiac capture or reduced pacing, patient demographics, patient history, or combinations thereof.

Clinicians have broad discretion in determining and implementing parameter settings of medical devices (e.g., CRT devices, etc.) but often follow published literature and guidelines or specific device limits. However, as recommendations change or new therapies, modes, parameters, or settings are introduced, it takes time for such literature or changes in such literature to become widely understood and adopted. For example, certain clinicians may have determined a specific set of parameter settings to optimize pacing in certain patient populations that differ from the previous literature or clinician training. Analysis of settings on a between-patient or between-clinician basis with respect to optimized pacing or capture can identify and determine different combinations of settings and distribute recommended sets of parameter settings more quickly than existing literature. Additionally, whereas clinicians focus on certain parameters, with access to a complete set of parameter settings across large numbers of patients, correlation between seemingly irrelevant parameters in combination with others can be determined that impact cardiac capture rates, improving pacing and cardiac resynchronization therapy, patient outcomes, device performance and efficiency, and communication of leading clinical data more quickly to clinician populations.

Artificial intelligence, particularly machine learning and other techniques, can effectuate the speed and analysis of identifying optimal settings and determining differences between different sets of parameter settings, in combination with physiologic information of the patient (such as determination of patient status, e.g., improving or worsening, etc.) or separate therefrom, taking into account rates of cardiac capture in specific patients or across populations. In addition, separate from tracking rates of cardiac capture for specific patients or patients having specific demographics, disease states, or patient conditions, rates for specific clinicians can be analyzed and determined to identify clinicians having more successful rates of cardiac capture across patients or patient groups.

For example, based on a specific desired output, such as optimizing cardiac resynchronization therapy by effectuating cardiac capture, etc., pacing parameter settings can be analyzed to identify or determine specific parameters or combinations thereof that are more likely correspond to unconfirmed or missed cardiac capture. In an example, although parameter settings often start from a default condition and are separately selectable and adjustable by a clinician, combinations of parameters often ideally move together. In an example, if one parameter is adjusted and a second is not, but adjustment of the second often provides optimal cardiac resynchronization therapy, detection of the second not being adjusted can trigger a recommendation to the clinician to adjust the second parameter. In other examples, such parameters can be adjusted automatically by the system with notice provided to the clinician of such change for review or approval.

In other examples, such as first programming after implant, or situations where patient physiologic information has not been previously recorded or is otherwise unavailable, proposed parameter settings can be recommended based on other information about the patient, such as age, gender, medications, co-morbidities, diagnosed conditions or disease states or progressions, or other information medical history information separate from sensed physiologic information. In this way, the first programmed values for a specific patient can differ from default values for all patients, potentially improving the speed of attaining optimal programming and reducing wasted resources associated with suboptimal operation.

Additionally, optimal parameter settings, or suggested combinations of parameter settings to optimize cardiac resynchronization therapy, may adjust over time, just as the parameters and settings themselves. In certain examples, optimal combinations of parameter settings or suggestions to optimize parameters settings for a particular patient can be provided to a clinician, such as during follow-up or report. In other examples, suboptimal pacing, including unconfirmed or confirmed missed cardiac capture, or a determined patient status or condition lower than a threshold or an expected improvement can trigger analysis and recommendation.

Periods of suboptimal therapy can be harmful to patients but may also lead to inefficient use of device resources including periods of stimulation by the device that may not provide a desired physiologic response, effectively wasting limited device resources. Identifying potentially less effective or ineffective parameter settings or combinations of parameter settings and providing a recommendation of one or more programming changes to improve pacing can result in a more efficient use of device resources while also improving patient therapy. Accordingly, identification of suboptimal settings and generating reprogramming recommendations can improve operation of the underlying hardware.

Parameter settings can be tracked, including patterns of changes across different patients and resulting impact on cardiac capture. Capture can include confirmed capture of one or more chambers, such as confirmed LV cardiac capture, confirmed RV cardiac capture, confirmed RA cardiac capture, or combinations thereof (e.g., confirmed Bi-V cardiac capture, including RV and LV, confirmed LV-only, etc.). In an example, confirmed cardiac capture in less than 98% of cardiac beats (or in other examples, less than 95% or less than 90%) over a period of time, such as a week, a day, a group of successive beats, etc., can trigger an alert or notification and analysis or re-analysis of device parameter settings. In other examples, a reduced trend of confirmed cardiac capture over time, or a sudden loss of cardiac capture below a threshold (e.g., from above 98% to lower than 90%, 80%, 50%, 20%, 10%, or to 0%, etc.) can trigger an alert or notification and analysis or re-analysis of device parameter settings. In other examples, all sets of parameter settings can be analyzed with respect to model parameter settings to identify suboptimal programming and suggest changes, in certain examples, additionally with respect to confirmed cardiac capture percentage. For example, if key opinion leaders change a model set of parameter settings, even in situations where a patient has confirmed cardiac capture at 98% or above, a notification can be provided to a clinician illustrating the differences and impact of such to the medical device and the patient.

In addition, during programming or reprogramming of medical devices, related parameter settings can be identified and grouped, such as to suggest corresponding changes (e.g., during initial programming, during follow-up sessions, etc.), where one parameter setting is changed, suggesting corresponding changes to one or more other programmer settings to optimize therapy to the patient. In addition, values of such parameter settings can be analyzed to suggest not only specific parameter settings to program or reprogram, but values of different parameters based upon proposed changes. For example, if changing one parameter by a first amount, a recommendation can be provided to change a second parameter by a second amount. In contrast, if changing the one parameter by an amount greater than the first amount, the recommendation can be changed to change the second or one or more other parameters by an among different than the second amount, greater or less, depending on the specific parameters.

A recommendation system, such as comprising one or more assessment circuits, can utilize artificial intelligence and machine learning models to identify corresponding changes, for example, corresponding to positive outcomes (or away from negative outcomes) with respect to confirmed cardiac capture percentages of cardiac beats in other patients. In certain examples, parameter settings by key opinion leaders can be weighed more heavily than parameter settings by other clinicians. In other examples, specific models based on parameter settings of key opinion leaders, including individual clinicians or groups of clinicians at the forefront of thought leadership in the field of cardiac rhythm management therapy, can be suggested, separate from other clinicians. Combinations of parameter settings (e.g., including patterns, values, etc.) can be identified and validated to optimize cardiac capture, and validated identified parameter settings can be recommended as optimal values to clinicians, such as when programming or reprogramming medical devices, including implantable medical devices configured to provide cardiac resynchronization therapy (e.g., CRT devices), etc. In certain examples, validation can include combinations of human and machine validation confirmed based on stored information and recorded patient outcomes.

In other examples, other potential suboptimal pacing scenarios can be improved using the systems and methods described herein, such as identifying suboptimal parameter settings associated with high pacing (e.g., a percentage of paced beats above a high or desired pacing threshold) in an implantable cardioverter defibrillator (ICD) (e.g., not a cardiac rhythm management device, but a defibrillator), or chronic overpacing in one or more ambulatory medical devices, where a patient condition could have changed such that existing settings, although resulting in cardiac capture, are not required any longer. For example, patient physiologic information, such as heart sound information (e.g., S1 amplitude, providing an indication of cardiac contractility of a ventricle, etc.) can be used to triage patients and determine whether periods of intrinsic activity should be allowed, or if one or more parameter settings should be adjusted or recommended commensurate with a change in the patient physiologic information or patient status determined using such information.

FIG. 2 illustrates an example 200 of a table 201 of parameter settings and a reduction 202 of different combinations of parameter settings. In an example, specific parameter settings for a medical device can be represented by changes or deviation from a mode (e.g., a most frequent, or in other examples another statistical parameter, such as an average, a median, etc.) for a respective parameter setting from other medical devices, other medical devices in similar patients (e.g., grouped by disease, physiologic information, patient status, or combinations thereof), or the same medical device in a patient but at a different times. For example, whereas each medical device has a set of parameter settings, respective medical devices also have different sets of parameter settings at different times.

A kernel is a type of function used in various AI and machine learning algorithms that enables an algorithm to operate in high-dimension space without computing the coordinates of the data in that space. For example, a kernel can compute a dot product of multiple vectors without having to compute the coordinates of the points in that space, saving computational resources. In an example, a kernel can be used to extract and store patterns in different sets of parameter settings of cardiac resynchronization therapy or other medical devices (e.g., an implantable cardioverter defibrillator (ICD), etc.) and to identify rules for different sets of parameters for specific sets or classes of devices performing similar functions, in certain examples, further tailored to specific patients based on physiologic information of such patients.

In an example, parameter settings for one medical device are illustrated in the table 201 across different office visits. The changes in settings are illustrated with a 1 or a 0 in the table 201. Columns or rows of the table 201, or in certain examples the table 201 itself, depending on organization, can be treated as vectors. In an example, columns A-D illustrate four different table entries. However, the four table entries A-D illustrate three parameter settings, P1-P3, with P1 having two states, reflecting different changes, for example, from a mode (most used value or setting for a particular parameter across a group of patients, such as in the training data, etc.) to a first value, illustrated as P1A, or from the mode to a second value, illustrated as P1B.

In a particular example, P1 can include an offset value, such as a sensed atrioventricular delay (AVD) offset value of 30, 40, or 50 ms, with a mode of 30 ms. PIA can represent a change from 30 ms to 40 ms, and PIB can represent a change from 30 ms to 50 ms. In other examples, additional table entries can be included, such as P1C illustrating a change from 40 ms to 50 ms, or other values, parameters, etc. P2 and P3 can include other parameter settings, such as particular sensing or tracking modes with 0 representing the mode (e.g., on or off) and 1 representing a change from the mode (e.g., off or on, respectively). As with P1 including P1A and P1B, P2 and P3 can also include other states as needed.

Parameter settings can be transformed into a table format or vectors, such as described herein (e.g., variance from a mode), and different combinations of settings and changes resulting therefrom (e.g., % of atrial or ventricular cardiac capture, etc.) can be evaluated to identify sets of parameter values or corresponding changes that represent the greatest positive impact, or positive impact above a threshold.

For example, changes in parameter settings can be evaluated to identify candidate rules or parameters values or combinations of parameter values that exceed a minimum support value (α) above a threshold (e.g., an upper percentile of response, etc.) to produce a reduction 202 of combinations of parameter settings. The minimum support value can be a function of frequency, impact, or combinations thereof.

In certain examples, upward closed analysis (or other analysis) can be used to reduce the overall work required to identify combinations of parameter values and associated rules. Upward closed analysis generally refers to a property where, if first and second parameter values are below a minimum support value (e.g., P(A)<α and P(B)<α), then any combination of parameters including the first and second parameter values will be below the minimum support value (P(A,B)<α). Based on such analysis, the reduction 202 illustrates that if P(D)<α, then all combinations including P(D) can be excluded from analysis. Similarly, if P(B,C)<α, then all combinations including P(B,C) can be excluded from analysis.

Although described herein with respect to upward closed analysis, in other examples other types of analysis can similarly be used, such as lower closed analysis or other order or lattice theory elements or properties, etc.

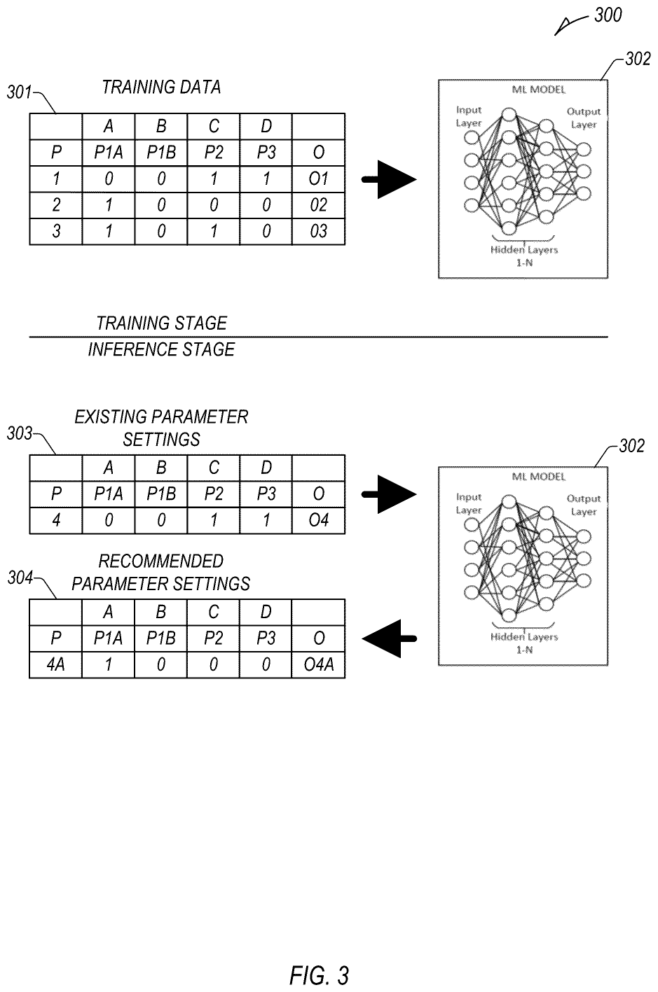

FIG. 3 illustrates a diagram 300 of an example machine learning model 302 trained to receive, as input, training data 301, such as parameter settings from one or more ambulatory medical devices, and in certain examples physiologic information associated with respective parameter settings, such as associated cardiac capture rates of different patients, to identify optimal or suboptimal parameter settings or combinations of parameter settings corresponding to successful cardiac resynchronization therapy, and to generate an output indicating proposed parameter settings to optimize cardiac resynchronization therapy or to reduce suboptimal, missed, or reduced pacing based on existing parameter settings.

In the training (or learning) stage of FIG. 3, the training data 301 is applied to a machine learning algorithm to train the machine learning model 302. The training data 301 comprises parameter settings (e.g., P1A, P1B, P2, P3, etc., such as described in FIG. 2) from a number of different medical devices (e.g., 1-3, etc.). Although illustrated as a small number of parameters settings (e.g., P1A, P1B, P2, P3) and medical devices (1-3), in practice the training data 301 can include substantially more parameter settings and medical devices, such as tens or hundreds of parameter settings and hundreds or thousands of medical devices. In certain examples, the same medical device can provide parameter settings at different times. In certain examples, each vector or set of parameter settings can include an outcome (O) or assessment information, such as patient physiologic information (e.g., cardiac capture information, etc.) or other information of or about a patient associated with the respective parameter settings. In certain examples, the assessment can be provided by or validated by a user, such as based on direct measurement of patient physiologic information, etc.

The machine learning model 302 can be trained using supervised learning, unsupervised learning, or reinforcement learning. Examples of machine learning model architectures and algorithms may include, for example, decision trees, neural networks, support vector machines, or deep neural networks, etc. Examples of deep neural networks can include a convolutional neural network (CNN), a recurrent neural network (RNN), a deep belief network (DBN), a long-term and short-term memory (LSTM) network, a transfer learning network, or a hybrid neural network comprising two or more neural network models of different types or different model configurations. The training of the machine learning model may be performed continuously or periodically, or in near real time as additional patient data are made available. The training involves algorithmically adjusting one or more model parameters or parameter settings, until the model being trained satisfies a specified training convergence criterion. The trained machine learning model 302 can establish a correspondence between parameter settings or combinations of parameter settings and cardiac capture.

In some examples, a machine learning model can be trained to analyze physiological signal data and detect cardiac capture or successful or optimal cardiac resynchronization therapy, or correspondingly, suboptimal cardiac resynchronization therapy. To train such a model, a dataset is assembled containing sample patient physiologic information annotated by medical experts to identify successful cardiac capture and correspondingly, unconfirmed cardiac capture or unsuccessful cardiac capture. This annotated training dataset is then used to train the machine learning model 302 using a supervised learning approach, such as by algorithmically adjusting internal parameters to map the input patient physiologic information to the expert-applied labels. Various machine learning algorithms can be used, such as described above. The training process continues until the model achieves a high accuracy in classifying optimal or sub-optimal cardiac resynchronization therapy.

The training and utilization of machine learning models to determine indications of cardiac capture and to identify parameters or combinations of parameters associated with optimal or sub-optimal cardiac resynchronization therapy will depend on the specific type of ambulatory medical device being used and the nature of the physiological signal data it collects. Once trained, the machine learning model can receive new parameter settings and patient physiologic information as input and automatically detect if the patterns reflect optimal or sub-optimal cardiac resynchronization therapy and to identify specific parameter settings or combinations of parameter settings to change to optimize cardiac resynchronization therapy. This allows advanced cardiac resynchronization therapy analysis without needing to hard-code detection criteria. The model can be periodically retrained on new data to optimize cardiac resynchronization therapy over time.

In the inference stage of FIG. 3, for example, after the machine learning model 302 has been trained to reliably analyze parameter settings, patient physiologic information, or combinations thereof, and to identify and suggest programming or reprogramming recommendations to optimize cardiac resynchronization therapy in a training dataset and/or an evaluation dataset, the trained machine learning model 302 can be deployed for use in a production setting. Accordingly, existing parameter settings 303 (P1A, P1B, P2, P3) of an ambulatory medical device (4) can be provided as input to the trained machine learning model 302, such as at a remote server computer, in certain examples including patient physiologic information (O4). The trained machine learning model 302 can generate one or more outputs, including recommended parameter settings 304, indicating reprogramming recommendations for the ambulatory medical device (4A) and in certain examples an indication of outcome expected by such reprogramming recommendations (O4A). In certain examples, the received patient physiologic information can be used as input to a rule-based recommendation engine for generating a recommendation to reprogram an ambulatory medical device, for example, when one or more parameter settings or combinations of parameter settings are identified by the machine learning analysis as suboptimal or having a high likelihood of resulting in suboptimal cardiac resynchronization therapy.

Consistent with some embodiments, a reprogramming recommendation may involve a recommendation to modify or reprogram the device to use one or more different settings in sensing events, activity, or physiologic information, such as one or more blanking periods, thresholds, etc., or in providing stimulation, such as one or more intervals, delays, stimulation amplitudes, selected electrodes or vectors, etc. In certain examples, the recommendation to modify or reprogram the device can include a recommendation to use one or more different sensitivity settings or modes different from the current sensitivity setting or mode. With some embodiments, each sensitivity setting, or sensitivity mode is associated with one or more predefined threshold values. Accordingly, when a device is reprogrammed to operate in a new sensitivity mode, one or more of the predefined threshold values can change, thereby increasing or decreasing the sensitivity for detecting a particular event or activity. The reprogramming recommendation may be presented via a user interface of a software application to a clinician, who may undertake the task of reprogramming the device for a patient. The clinician can then evaluate the recommendation and reprogram the ambulatory medical device accordingly. This allows the clinician to validate any proposed changes to the device based on their expert judgment. In other examples, the reprogramming recommendation can be automatically applied within limits, such as previously validated by the clinician, etc., or directed based on physiologic response of the patient, such as determined by one or more patient response metrics indicative of patient response to different cardiac rhythm management therapy.

Embodiments of the present invention provide numerous technical advantages for optimizing programming of and therapy delivery by ambulatory medical devices. By periodically evaluating collected physiological signal data using advanced machine learning models, embodiments of the invention enable closed-loop optimization of operation of the ambulatory medical device over time. This allows enhancing operation of the ambulatory medical device compared to relying solely on static detection settings programmed at implantation. The machine learning analysis can identify suboptimal parameter settings missed by the ambulatory medical device or the clinician programming the ambulatory medical device and recommend adjustments to improve operation when appropriate. The system can adapt parameter settings to the individual patient commensurate with patient status or therapy efficacy, providing more accurate therapy and without requiring constant manual reprogramming, reducing workload for clinicians while optimizing device performance and increasing the speed of training and sharing updated protocols and guidance.

Parameter settings differ based on the type of medical devices and in certain examples can include different modes of therapy. For example, different modes of cardiac resynchronization pacing include DDD and DDDR pacing, among others. The first “D” in DDD pacing represents dual (D) chamber (atrium and ventricle) pacing, the second “D” represents dual (D) chamber sensing, and the third “D” represents dual (D) chamber response to sensing in coordinating contraction of the heart and improve cardiac function. The additional “R” in DDDR pacing represents rate (R) modulation, where the medical device can adjust the pacing rate based on physiologic information indicating activity or need (e.g., activity, breathing rate, etc.).

Parameter settings can include, among others: Sensed Atrioventricular Delay Offset (SenAVDIyOffset or SAVDO), which adjusts the delay after a sensed atrial event; Atrioventricular Dynamic Minimum (AVDynMin or AVDM), which sets the minimum dynamic atrioventricular delay; Atrioventricular Delay Fixed (AVDlyFix or AVDF), which is a fixed atrioventricular delay setting; Atrioventricular Dynamic Maximum (AVDynMax or AVDM), which defines the maximum dynamic atrioventricular delay; Lower Rate Interval (LRLIntvl or LRLI), which sets the minimum pacing rate for the device; Left Ventricular Offset (LVOffset or LVO), which adjusts the timing of left ventricular pacing in relation to right ventricular pacing; Atrioventricular Dynamic Enable (AVDynEnbl or AVDE), which enables dynamic adjustment of the AVD; and Maximum Tracking Rate Interval (MTRIntvl or MTRI), the fastest interval at which the device will track atrial rates.

Additional parameter settings can include: Atrial Tachy Response Mode (ATRMode or ATRM), which defines the operational mode of the atrial channel; Biventricular Trigger Enable (BiVTrigEnbl or BVTE), which activates the biventricular pacing trigger; Ventricular Tachycardia Zone Rate (VTZoneRate or VTZR), which sets the rate threshold for detecting ventricular tachycardia; Atrial Tachy Response Trigger Rate (ATRTrigRt or ATRTR), which is the rate at which Atrial Tachy Response Mode is triggered; Maximum Sensor Rate Interval (MSRIntvl or MSRI), the maximum rate at which the device will pace in response to sensor input; Ventricular Tachycardia 1 Zone Rate (VT1ZoneRate or VT1ZR), which specifies the rate for a particular zone of ventricular tachycardia detection; Number of Ventricular Zones (NumVZones or NVZ), which determines how many zones are used for ventricular tachyarrhythmia detection; Ventricular Fibrillation Zone Rate (VFZoneRate or VFZR), which sets the rate threshold for detecting ventricular fibrillation; Atrial Tachy Response Ventricular Rate Regulation Response (ATRVRRResp or ATRVRRR), a setting that adjusts the ventricular pacing rate in response to atrial rate; Atrial Tachy Response Biventricular Trigger Enable (ATRBiVTrigEnbl or ATRBVTE), which allows for biventricular pacing in response to atrial rate; Atrial Tachy Response Lower Rate Limit (ATRLRL), which sets the minimum pacing rate in ATR mode; Tachycardia Mode (TachyMode or TM), which defines the operational mode for tachycardia detection and therapy; Respiration Rate Trend Enable (RRTenable or RRT), which activates the respiration rate tracking feature; Atrial Tachy Response Pacing Chamber (ATRPaceCham or ATRPC), which specifies the chamber to be paced in atrial tachy mode; and Sensing Mode (SenseMode), which determines how the device senses cardiac events.

### CRT vs. MSP Mode Switch

Certain heart failure patients respond to (e.g., benefit from) multi-site pacing therapy that do not respond to traditional (non-multi-site pacing) cardiac resynchronization therapy. Other patients do not respond to either. Pacing therapies are frequently evaluated, such as to ensure that the applied pacing therapy is providing some benefit to the patient, to determine whether or not the pacing therapy should be adjusted, or to determine if the pacing therapy should be transitioned. For example, a study by Samir Saba et al. titled “Usefulness of Multisite Ventricular Pacing in Nonresponders to Cardiac Resynchronization Therapy” in The American Journal of Cardiology, Feb. 1, 2022 (herein “the Saba study”) found that 25% to 40% of heart failure patients with myocardial dysfunction did not respond to conventional cardiac resynchronization therapy (e.g., bi-ventricular pacing therapy) by evaluation at 6 months post implant, but also found that over half (51.3%) of non-responders to cardiac resynchronization therapy at 6 months did subsequently respond to 6 months of multi-site pacing therapy in the left ventricle by evaluation at 12 months post implant.

Although the transition from CRT to multi-site pacing therapy in the left ventricle was substantially complication free physiologically, the switch to multi-site pacing therapy from conventional CRT can provide at least some cardiac stress, but also requires additional resources from the implantable medical device. In certain estimates, implementing multi-site pacing therapy in a device capable of multi-site pacing therapy and CRT reduces the estimated life of the device by 11-13%. Even a single six-month evaluation of multi-site pacing therapy on an IMD can impact the usable life of the IMD by a relatively substantial and often unnecessary amount.

In certain examples, physiologic information sensed from the patient in one or more time periods can be used to determine one or more multi-site pacing response metrics, and that such one or more determined multi-site pacing response metrics can be used to provide an alert or notification or otherwise control transition between different medical device modes (e.g., a first stimulation mode, a second stimulation mode, etc.), including, in certain examples, to: (1) control transition of a non-multi-site pacing therapy mode to a multi-site pacing therapy mode; (2) control transition of the multi-site pacing therapy mode to the non-multi-site pacing therapy mode; or (3) enable or disable the multi-site pacing therapy mode.

Moreover, physiologic information sensed from the patient subsequent to implementing a stimulation mode can be used to determine a multi-site pacing response metric configured to determine an indication of a predicted patient response to the stimulation mode, such that, in certain examples, a stimulation mode can be evaluated without entering the stimulation mode. For example, specific physiologic information or combinations of physiologic information can be determined to evaluate a stimulation mode or to predict a positive patient response to a particular stimulation mode, such as using a response to another stimulation mode, etc. In an example, physiologic information sensed or detected during a CRT mode can be used to determine if a patient is likely to respond to (e.g., benefit from) a multi-site pacing therapy mode before the multi-site pacing therapy mode is implemented or enabled.

In an example, impedance information (e.g., ITTI), respiration information (e.g., RSBI), or a combination thereof over one or more time periods can be used to determine an indication that a patient will respond to a multi-site pacing therapy mode. A first patient response metric, p1, (e.g., a multi-site pacing response metric) can be determined as follows:

$$( α \times ITTI ) + ( β \times RSBI ) \qquad ( 1 )$$

In equation (1), ITTI is a measure of patient intrathoracic impedance, RSBI is a measure of patient respiration rate (RR) or frequency to a measure of tidal volume (TV) (e.g., RR/TV, etc.), and α and β are variables. In other examples, the patient response metric can be determined using only one of ITTI or RSBI information, or as different functions of one or more of such physiologic parameters in combination with one or more other physiologic parameters.

In other examples, other patient response metrics can be determined using other physiologic information, such as heart sound information (e.g., S3/S1, etc.), RSBI information, ITTI information, etc. For example, S3/S1, is particularly well correlated to determining if a patient is likely to respond to a CRT mode (or CRT mode or MSP therapy mode), in contrast to being a non-responder (e.g., there is a difference in heart sound information between responders and non-responders). RSBI information is similarly well correlated. A second patient response metric, p2, (e.g., a CRT pacing response metric) can be determined as follows:

$$( α \times ( S 3 / S 1 ) ) + ( β \times RSBI ) \qquad ( 2 )$$

In equation (2), S3 is a third heart sound parameter (e.g., an amplitude or energy of the third heart sound, etc.), S1 is a first heart sound parameter (e.g., an amplitude or energy of the first heart sound, etc.), RSBI is a measure of patient respiration rate (RR) or frequency to a measure of tidal volume (e.g., RR/TV, etc.), and a and B are variables. In other examples, the patient response metric can be determined using a combination of this or other heart sound information (e.g., other than S3/S1, etc.), using only one of heart sound or RSBI information, using information over different time periods, or as a combination with one or more other physiologic parameters.

In other examples, one or more other patient response metrics can be determined using sensed or received physiologic information of the patient, or combinations of sensed or received physiologic information of the patient, such as one or more of night heart rate information, activity information, determination of patient rest (e.g., a lack of activity, etc.), a multi-sensor HeartLogic index, a slope of a minute ventilation (MV) signal, determined AVD parameters, pacing thresholds in or in response to the different therapies or modes, continuous ECG measurements, impedance measurements, occurrence or detection of adverse events (e.g., arrhythmia, etc.), etc. The HeartLogic index is a composite heart failure risk indication determined using a combination of different physiologic information, including heart sound information (including S1 and S3), respiration rate and volume information, ITTI information, heart rate information (e.g., particularly night heart rate determined between midnight and 6am with respect to the patient), and daily patient activity information (e.g., daily hours above an activity threshold). Different modes or parameter settings can be implemented for a time period (e.g., 1 week, 1 month, etc.), the patient response metric can be determined in response thereto, and the therapy can be adjusted accordingly, with reporting and alerts coincident to changes in determined patient response metrics, with changes in parameter settings, or in response to changes exceeding one or more thresholds. In certain examples, patient response metrics can include a comparison of a determined patient response metric pre and post change and can trigger further adjustment or a report to a clinician for review.

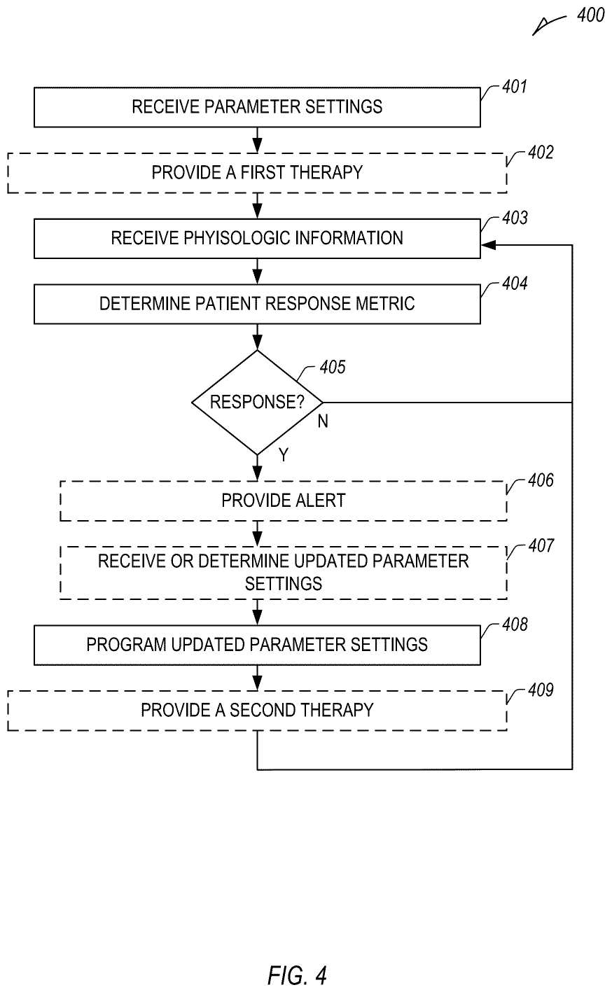

FIG. 4 illustrates an example method 400 of evaluating patient response to cardiac rhythm management therapy by an implantable medical device and for remotely reprogramming the implantable medical device.

In an example, an implantable medical device, once implanted and in certain examples after a recovery period, can begin in a first mode, such as a cardiac resynchronization therapy mode or one or more other therapy modes having different parameter settings or a monitoring mode, depending on the patient or clinician. In certain examples, a stimulation circuit can generate and provide one or more stimulation signals in one or more stimulation modes, and the assessment circuit can be configured to control the stimulation circuit, such as to adjust one or more parameters or transition between different therapy modes, etc.

At step 401, parameter settings can be received, such as using a signal receiver circuit or an assessment circuit. The parameter settings can include parameter settings from a clinician during a programming or reprogramming session, existing parameter settings of an implantable medical device, or other parameter settings of or proposed for the implantable medical device. In an example, the parameter settings can include settings indicative of a state or mode of the implantable medical device and can be received from an implantable medical device or a remote or external device configured to program the implantable medical device.

At step 402, a therapy, such as a first therapy, can be provided to the patient, such as using the implantable medical device according to the received parameter settings. For example, parameter settings in a first time period can be representative of a first cardiac rhythm management therapy, implemented at a time of implant or later, such as after a monitoring mode, after a recovery period, or after one or more previous reprogramming instructions. Parameter settings can subsequently be received in a second time period or one or more other time periods subsequent to the first time period, each representative of a specific therapy having defining characteristics, such as a mode or one or more parameter settings.

At step 403, physiologic information of the patient having the implantable medical device can be received from one or more sensors of the implantable medical device or one or more other ambulatory medical devices associated with the patient, such as using the signal receiver circuit. The physiologic information can include one or more types of physiologic information sensed using one or sensors of an implantable medical device, such as described herein. In one example, the received physiologic information can include respiration information sensed using one or both of an accelerometer or an impedance sensor. In other examples, the received physiologic information can include other information, such as heart sound information, activity information, heart rate information, etc., sensed using one or more sensors of an implantable medical device.

At step 404, a patient response metric can be determined using the received physiologic information, such as described herein, in certain examples, using the assessment circuit. In other examples, the patient response metric can be determined as a function of one or more types or values of physiologic information of the patient, such as one or more of respiration variability information or daily activity information, or in certain examples, heart sound information, RSBI information, or combinations or permutations thereof. The patient response metric can be indicative of a patient status for the respective therapy, mode, or parameter settings of a respective time period from which the patient response metric is based. In certain examples, the determined patient response metric can indicate a need for CRT, MSP therapy, or combinations thereof, or that the patient is a likely responder or non-responder to CRT or MSP therapy. In other examples, the determined patient response metric can be used to control transition between therapy modes, such as to transition into, between, or out of one or more of a CRT mode and a MSP therapy mode, etc.

At step 405, a value of the patient response metric can be compared to one or more thresholds, such as using the assessment circuit, for example. In certain examples, the threshold can include a relative threshold based on a patient response metric in one or more previous time periods, a patient baseline, or an expected positive or negative trend of the patient response metric over a time period of the respective therapy, such as with respect to a previous time period or an expected value, for example, selected or determined by a clinician or with reference to the patient or a group of patients. In certain examples, at least one of the one or more thresholds can include alert thresholds indicative of a worsening patient status requiring clinician review. In other examples, the one or more thresholds can be indicative of a worsening patient status, such as to trigger a therapy adjustment, but below the alert threshold.

If the value of the patient response metric exceeds the one or more thresholds, indicating a worsening patient status or condition with respect to the threshold, the method can proceed to step 406. If the value of the patient response metric does not exceed the threshold, indicating an improving patient status, monitoring can proceed and the method can return to step 403 or one or more other steps. In an example, comparison to the one or more thresholds can occur after a period to time, and in certain examples a report can be determined based on the determined patient response metric for the period of time. In other examples, comparison to the one or more thresholds can occur in real-time, irrespective of time periods, with an alert provided if the determined patient response metric exceeds the threshold.

At step 406, an alert can be provided, such as by the assessment circuit, for example, if the determined patient response metric exceeds the one or more thresholds, or in other examples if one or more reports of the determined patient status are available for review or transmission, if one or more changes in patient physiologic information or are determined or detected, such as above a threshold, etc. In an example, an output can be provided of the alert to a user interface for display to a user or to another circuit to control or adjust a process or a function of an implantable or ambulatory medical device, such as to adjust a therapy, mode, parameter settings, or follow-up schedule associated with the patient, a clinician, etc., with each of such adjustments at least partially dependent on the value of the determined patient response metric or the amount that the determined patient response metric exceeds the one or more thresholds.

At step 407, updated parameter settings can be received or determined, such as by applying one or more pre-trained machine learning models by one or more assessment circuits of the patient management system, including, for example, one or more remote devices, etc. In other examples, updated parameter settings can be received from a clinician upon review of the determined patient response metric. In an example, the alert to the clinician can include recommended updated parameter settings for clinician review, and the clinician can instruct updated parameter settings to be applied responsive thereto.

In an example, the one or more pre-trained machine learning models can be trained, in certain examples, such as described herein. For example, the determined patient response metric and parameter settings can be input into one or more pre-trained machine learning models trained to compare the received parameter settings to stored model parameter settings from one or more other implantable medical devices corresponding to one or more other patients and to identify one or more differences between the parameter settings of the implantable medical device and the stored model parameter settings of the one or more other implantable medical devices and different determined patient response metrics. Upon obtaining an output from the one or more pre-trained machine learning models indicating the identified one or more differences between the parameter settings of the implantable medical device and parameter settings of the one or more other implantable medical devices, a programming recommendation can be generated for the implantable medical device to improve cardiac capture for the patient based on the identified one or more differences.

In other examples, if the determined patient response metric indicates that the existing therapy is stable or changing, etc., one or more modes or functions of the implantable or ambulatory medical device can be altered to increase or decrease a power consumption or sensing or storage capability of the implantable or ambulatory medical device. For example, one or more hardware limitations can be adjusted, such as to, among others: sense or receive more or less physiologic information of the patient; increase or decrease communication frequency between the implantable or ambulatory medical device and an external device (e.g., remote device, programmer, etc.), such as to increase or reduce the frequency of patient monitoring, etc.; switch to a different power or resource intensive monitoring algorithm; etc. In an example, if the determined patient response metric is stable after an extended period of time (e.g., greater than 6 months, greater than one year, greater than two years, five years, etc.), programming recommendations can be provided to a clinician for review, or implemented automatically, to reduce power consumption or extend device lifespan without negatively impacting the patient. In other examples, if the determined patient response metric has been stable for an extended period of time but then starts to drift (e.g., starts to slowly decrease, but not yet triggering any alert thresholds, etc.), one or more modes or parameter settings can be adjusted to determine if different parameter settings can reduce drift or improve patient condition before a subsequent follow-up. In certain examples, changes can be prohibited if the determined patient response metric is not stable at a time of a proposed change.

At step 408, updated parameter settings can be programmed to the implantable medical device, such as by the assessment circuit, through one or more communication circuits, etc., to a user or process, such as providing an output of updated parameter settings to a user interface for display to the user or to a control circuit to control or adjust the process or function of the medical device system, etc. The updated parameter settings can be stored, such as using the assessment circuit, and transmitted, by control of the assessment circuit or using one or more communication circuits, etc., such as to one or more additional processes or components, such as an output circuit (e.g., a display, a controller for a display, etc.). In certain examples, the updated parameter settings from a clinician or automated process can be programmed to the implantable medical device without requiring an in-clinic follow-up appointment by the patient.

At step 409, a second therapy can be provided to the patient based on the updated parameter settings, the determined patient response metric, or one or more other measures, values, parameter settings, or metrics, such as described herein. In an example, the second therapy can be provided, in response to the detected worsening patient response metric at step 405, such as to confirm a patient worsening condition (e.g., and not sub-optimal parameter settings for the current patient condition) prior to scheduling a follow-up (e.g., an in-person or in-clinic follow-up appointment, etc.). In certain examples, if, following the second therapy at step 409, a subsequent determined patient response metric indicates an improving patient condition, such as compared to the determined patient response metric in response to the first therapy, a follow-up (e.g., an in-person follow-up) may not yet be necessary (e.g., additional parameter settings can be tried, an alert can be provided to a clinician for review, etc.). In other examples, if the determined patient response metric in response to the second therapy 409 is worse, the same as, or similar (e.g., within a threshold amount or percentage, etc.), a follow-up can be scheduled or an existing follow-up schedule can be modified (e.g., to occur sooner, etc.), such as by one or more circuits (e.g., an assessment circuit, a scheduling circuit, etc.) or components of an external system (e.g., an external device, a remote device, etc.), etc.

Although illustrated as a method from step 401 through step 409, in certain examples, one or more steps are options, and in other examples, different combinations or permutations of these or other steps or examples can be combined to form other methods or processes, which is also applicable to other examples discussed herein. For example, a change from a first therapy to a second therapy or a comparison to a threshold can correspond to time periods illustrated in FIG. 1 associated with a report, a change, or an initial or subsequent follow-up.

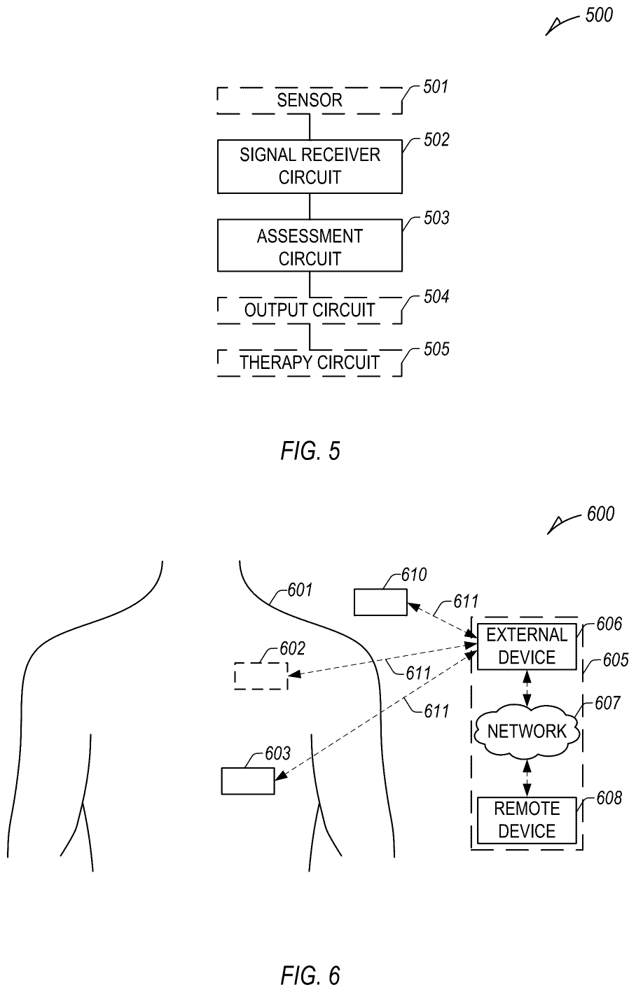

FIG. 5 illustrates an example system 500 (e.g., a medical device system). In an example, one or more aspects of the example system 500 can be a component of, or communicatively coupled to, a medical device, such as an implantable medical device (IMD), an insertable cardiac monitor (ICM), an ambulatory medical device (AMD), etc. The system 500 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 500 can include a single medical device or a plurality of medical devices implanted in a patient's body or otherwise positioned on or about the patient to monitor patient physiologic information of the patient using information from one or more sensors, such as a sensor 501. In an example, the sensor 501 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 500 can include a signal receiver circuit 502 and an assessment circuit 503. The signal receiver circuit 502 can be configured to receive physiologic information of a patient (or group of patients) from the sensor 501. The assessment circuit 503 can be configured to receive information from the signal receiver circuit 502, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 503 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 500 can include an output circuit 504 configured to provide an output to a user, or to cause an output to be provided to a user, such as through an output, a display, or one or more other user interface, the output including a score, a trend, an alert, or other indication. In other examples, the output circuit 504 can be configured to provide an output to another circuit, machine, or process, such as a therapy circuit 505 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more cardiac resynchronization therapy parameters, drug delivery, dosage determinations or recommendations, etc. In an example, the therapy circuit 505 can include one or more of a stimulation control circuit, a cardiac stimulation circuit, a neural stimulation circuit, a dosage determination or control circuit, etc. In other examples, the therapy circuit 505 can be controlled by the assessment circuit 503, or one or more other circuits, etc. In certain examples, the assessment circuit 503 can include the output circuit 504 or can be configured to determine the output to be provided by the output circuit 504, while the output circuit 504 can provide the signals that cause the user interface to provide the output to the user based on the output determined by the assessment circuit 503.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

In an example, following a change in parameter settings or therapy, a high-power mode of the medical device can be enforced for a monitoring period following the change, such as to determine one or more patient response metrics or patient status in response to the change in the high-power mode, in contrast to, for example, a low-power or ambulatory monitoring mode. In an example, the high-power mode can also be enforced in a time period prior to a follow-up appointment, in certain examples, consistent with the lock-out period otherwise described herein.

FIG. 6 illustrates an example patient management system 600 and portions of an environment in which the patient management system 600 may operate. The patient management system 600 can perform a range of activities, including remote patient monitoring and diagnosis of a disease condition. Such activities can be performed proximal to a patient 601, such as in a patient home or office, through a centralized server, such as in a hospital, clinic, or physician office, or through a remote workstation, such as a secure wireless mobile computing device.

The patient management system 600 can include one or more medical devices, an external system 605, and a communication link 611 providing for communication between the one or more ambulatory medical devices and the external system 605. The one or more medical devices can include an ambulatory medical device (AMD), such as an implantable medical device (IMD) 602, a wearable medical device 603, or one or more other implantable, leadless, subcutaneous, external, wearable, or medical devices configured to monitor, sense, or detect information from, determine physiologic information about, or provide one or more therapies to treat various conditions of the patient 601, such as one or more cardiac or non-cardiac conditions (e.g., dehydration, sleep disordered breathing, etc.).

In an example, the implantable medical device 602 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 601. In another example, the implantable medical device 602 can include a monitor implanted, for example, subcutaneously in the chest of patient 601, the implantable medical device 602 including a housing containing circuitry and, in certain examples, one or more sensors, such as a temperature sensor, etc.

Cardiac rhythm management devices, such as insertable cardiac monitors (ICMs), pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 602 can include an assessment circuit configured to detect or determine specific physiologic information of the patient 601, or to determine one or more conditions or provide information or an alert to a user, such as the patient 601 (e.g., a patient), a clinician, or one or more other caregivers or processes, such as described herein. The implantable medical device 602 can alternatively or additionally be configured as a therapeutic device configured to treat one or more medical conditions of the patient 601. The therapy can be delivered to the patient 601 via the lead system and associated electrodes or using one or more other delivery mechanisms. The therapy can include delivery of one or more drugs to the patient 601, such as using the implantable medical device 602 or one or more of the other ambulatory medical devices, etc. In some examples, therapy can include cardiac resynchronization therapy for rectifying dyssynchrony and improving cardiac function in heart failure patients. In other examples, the implantable medical device 602 can include a drug delivery system, such as a drug infusion pump to deliver drugs to the patient for managing arrhythmias or complications from arrhythmias, hypertension, hypotension, or one or more other physiologic conditions. In other examples, the implantable medical device 602 can include one or more electrodes configured to stimulate the nervous system of the patient or to provide stimulation to the muscles of the patient airway, etc.

The wearable medical device 603 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 605 can include a dedicated hardware/software system, such as a programmer, a remote server-based patient management system, or alternatively a system defined predominantly by software running on a standard personal computer. The external system 605 can manage the patient 601 through the implantable medical device 602 or one or more other ambulatory medical devices connected to the external system 605 via a communication link 611. In other examples, the implantable medical device 602 can be connected to the wearable medical device 603, or the wearable medical device 603 can be connected to the external system 605, via the communication link 611. This can include, for example, programming or reprogramming the implantable medical device 602 with different parameter settings, such as to perform one or more of acquiring physiologic data, performing at least one self-diagnostic test (such as for a device operational status), analyzing the physiologic data, or optionally delivering or adjusting a therapy for the patient 601. Additionally, the external system 605 can send information to, or receive information from, the implantable medical device 602 or the wearable medical device 603 via the communication link 611. Examples of the information can include real-time or stored physiologic data from the patient 601, diagnostic data, such as detection of patient hydration status, hospitalizations, responses to therapies delivered to the patient 601, or device operational status of the implantable medical device 602 or the wearable medical device 603 (e.g., battery status, lead impedance, etc.). The communication link 611 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 605 can include an external device 606 in proximity of the one or more ambulatory medical devices, and a remote device 608 in a location relatively distant from the one or more ambulatory medical devices, in communication with the external device 606 via a communication network 607. Examples of the external device 606 can include a medical device programmer. The remote device 608 can be configured to evaluate collected patient information and provide alert notifications, among other possible functions. In an example, the remote device 608 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 608 can receive data from multiple patients. The data can be collected by the one or more ambulatory medical devices, among other data acquisition sensors or devices associated with the patient 601. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

In an example, similar to the alert notifications discussed above, the external system 605 or one or more components thereof (e.g., the external device 606, the remote device 608, an assessment circuit, etc.) can be configured to schedule one or more follow-up appointments or adjust a schedule of one or more follow-up appointments for the patient such as in response to one or more alert notifications or other determinations, per a request of a clinician, etc.

The remote device 608 may additionally include one or more locally configured clients or remote clients securely connected over the communication network 607 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 608, including the server and the interconnected clients, may also execute a follow-up scheme by sending follow-up requests to the one or more ambulatory medical devices, or by sending a message or other communication to the patient 601 (e.g., the patient), clinician or authorized third party as a compliance notification.

The communication network 607 can provide wired or wireless interconnectivity. In an example, the communication network 607 can be based on the Transmission Control Protocol/Internet Protocol (TCP/IP) network communication specification, although other types or combinations of networking implementations are possible. Similarly, other network topologies and arrangements are possible.

One or more of the external device 606 or the remote device 608 can output the detected medical events to a system user, such as the patient or a clinician, or to a process including, for example, an instance of a computer program executable in a microprocessor. In an example, the process can include an automated generation of recommendations for therapy, or a recommendation for further diagnostic test or treatment. In an example, the external device 606 or the remote device 608 can include a respective display unit for displaying the physiologic or functional signals, or alerts, alarms, emergency calls, or other forms of warnings to signal the detection of arrhythmias. In some examples, the external system 605 can include an external data processor configured to analyze the physiologic or functional signals received by the one or more ambulatory medical devices. Computationally intensive algorithms, such as machine-learning algorithms, can be implemented in the external data processor to process the data retrospectively to detect cardiac arrhythmias.

Portions of the one or more ambulatory medical devices or the external system 605 can be implemented using hardware, software, firmware, or combinations thereof. Portions of the one or more ambulatory medical devices or the external system 605 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 610 can be configured to send information to or receive information from one or more of the ambulatory medical devices or the external system 605 using the communication link 611. In an example, the one or more ambulatory medical devices, the external device 606, or the remote device 608 can be configured to control one or more parameters of the therapy device 610. The external system 605 can allow for programming the one or more ambulatory medical devices and can receives information about one or more signals acquired by the one or more ambulatory medical devices, such as can be received via a communication link 611. The external system 605 can include a local external implantable medical device programmer. The external system 605 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

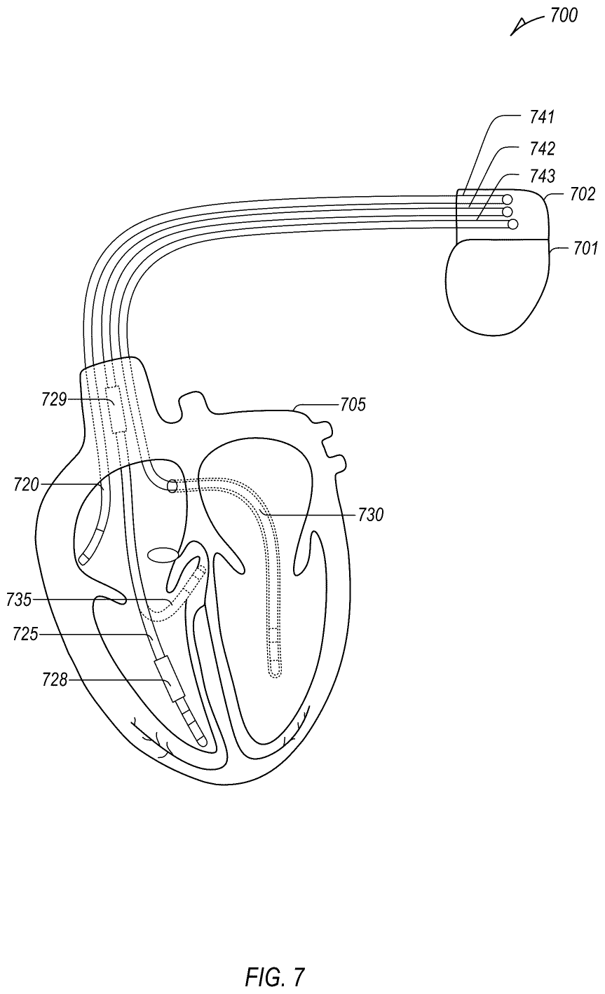

FIG. 7 illustrates an example implantable medical device (IMD) 700 electrically coupled to a heart 705, such as through one or more leads coupled to the implantable medical device 700 through one or more lead ports, such as first, second, or third lead ports 741, 742, 743 in a header 702 of the implantable medical device 700. In an example, the implantable medical device 700 can include an antenna, such as in the header 702, configured to enable communication with an external system (e.g., the external system 605) and one or more electronic circuits (e.g., the assessment circuit 503) in a hermetically sealed housing (CAN) 701.

The implantable medical device 700 may include an implantable cardiac monitor (ICM), pacemaker, defibrillator, cardiac resynchronizer, or other subcutaneous implantable medical device or cardiac rhythm management (CRM) device configured to be implanted in a chest of a subject, having one or more leads to position one or more electrodes or other sensors at various locations in or near the heart 705, such as in one or more of the atria or ventricles. Separate from, or in addition to, the one or more electrodes or other sensors of the leads, the implantable medical device 700 can include one or more electrodes or other sensors (e.g., a pressure sensor, an accelerometer, a gyroscope, a microphone, etc.) powered by a power source in the implantable medical device 700. The one or more electrodes or other sensors of the leads, the implantable medical device 700, or a combination thereof, can be configured detect physiologic information from, or provide one or more therapies or stimulation to, the patient.

Implantable devices can additionally include a leadless cardiac pacemaker (LCP), small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 705 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple LCP devices can communicate between themselves, or one or more other implanted or external devices.

The implantable medical device 700 can include one or more electronic circuits configured to sense one or more physiologic signals, such as an electrogram or a signal representing mechanical function of the heart 705. In certain examples, the CAN 701 may function as an electrode such as for sensing or pulse delivery. For example, an electrode from one or more of the leads may be used together with the CAN 701 such as for unipolar sensing of an electrogram or for delivering one or more pacing pulses. A defibrillation electrode (e.g., the first defibrillation coil electrode 728, the second defibrillation coil electrode 729, etc.) may be used together with the CAN 701 to deliver one or more cardioversion/defibrillation pulses.

In an example, the implantable medical device 700 can sense impedance such as between electrodes located on one or more of the leads or the CAN 701. The implantable medical device 700 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 700 can be configured to inject current between an electrode on one or more of the first, second, third, or fourth leads 720, 725, 730, 735 and the CAN 701, and to sense the resultant voltage between the same or different electrodes and the CAN 701.

The implantable medical device 700 can integrate one or more other physiologic sensors to sense one or more other physiologic signals, such as one or more of heart rate, heart rate variability, intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are possible.

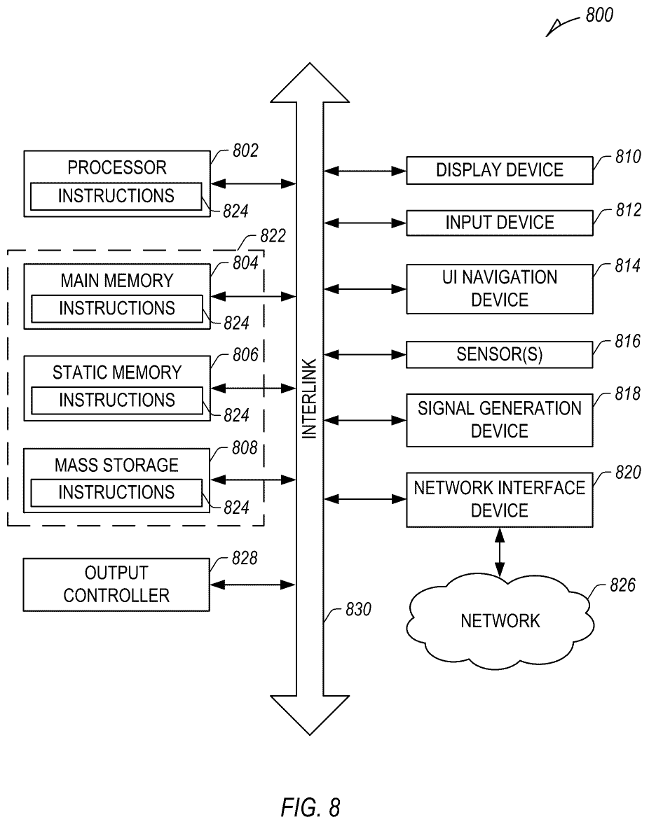

FIG. 8 illustrates a block diagram of an example machine 800 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 800. Circuitry (e.g., processing circuitry, an assessment circuit, etc.) is a collection of circuits implemented in tangible entities of the machine 800 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 800 follow.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 800 (e.g., computer system) may include a hardware processor 802 (e.g., a central processing unit (CPU), a graphics processing unit (GPU), a hardware processor core, or any combination thereof), a main memory 804, a static memory 806 (e.g., memory or storage for firmware, microcode, a basic-input-output (BIOS), unified extensible firmware interface (UEFI), etc.), and mass storage 808 (e.g., hard drive, tape drive, flash storage, or other block devices) some or all of which may communicate with each other via an interlink 830 (e.g., bus). The machine 800 may further include a display unit 810, an input device 812 (e.g., a keyboard), and a user interface (UI) navigation device 814 (e.g., a mouse). In an example, the display unit 810, input device 812, and UI navigation device 814 may be a touch screen display. The machine 800 may additionally include a signal generation device 818 (e.g., a speaker), a network interface device 820, and one or more sensors 816, such as a global positioning system (GPS) sensor, compass, accelerometer, or one or more other sensors. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 802, the main memory 804, the static memory 806, or the mass storage 808 may be, or include, a machine-readable medium 822 on which is stored one or more sets of data structures or instructions 824 (e.g., software) embodying or utilized by any one or more of the techniques or functions described herein. The instructions 824 may also reside, completely or at least partially, within any of registers of the hardware processor 802, the main memory 804, the static memory 806, or the mass storage 808 during execution thereof by the machine 800. In an example, one or any combination of the hardware processor 802, the main memory 804, the static memory 806, or the mass storage 808 may constitute the machine-readable medium 822. While the machine-readable medium 822 is illustrated as a single medium, the term “machine-readable medium” may include a single medium or multiple media (e.g., a centralized or distributed database, and/or associated caches and servers) configured to store the one or more instructions 824.

The term “machine-readable medium” may include any medium that is capable of storing, encoding, or carrying instructions for execution by the machine 800 and that cause the machine 800 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may be further transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 800, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for evaluating patient response to cardiac rhythm management therapy by an implantable medical device and for remotely reprogramming the implantable medical device, the system comprising:
one or more processors; and
one or more memory devices storing instructions, which when executed by the one or more processor, cause the one or more processors to perform operations comprising:
receiving physiologic information of a patient from the implantable medical device, including physiologic information of the patient in a first time period responsive to the implantable medical device providing a first cardiac rhythm management therapy to the patient;
determining, using the physiologic information of the patient in the first time period, a first patient response metric indicative of patient response to the first cardiac rhythm management therapy;
generating, based on determining that a value of the first patient response metric exceeds a first threshold, a reprogramming recommendation for the implantable medical device including a second cardiac rhythm management therapy based on the first patient response metric; and
remotely reprogramming the implantable medical device to provide a second cardiac rhythm management therapy to the patient in a second time period subsequent to the first time period,
wherein the first time period includes a post-implant time period following implant of the implantable medical device in the patient.

2. The system of claim 1, wherein the operations comprise:
receiving an indication of a time of implant of the implantable medical device in the patient, wherein the post-implant time period comprises a pre-determined time period from the time of implant of the implantable medical device.

3. The system of claim 2, wherein the pre-determined time period includes a time period of 2 to 4 weeks after the time of implant of the implantable medical device or after a recovery period after the time of implant of the implantable medical device.

4. The system of claim 1, wherein the operations comprise:
receiving physiologic information of the patient from the implantable medical device, including physiologic information of the patient in the second time period responsive to the implantable medical device providing the second cardiac rhythm management therapy to the patient;
determining, using the physiologic information of the patient in the second time period, a second patient response metric indicative of patient response to the second cardiac rhythm management therapy;
generating, based on determining that a value of the second patient response metric exceeds a second threshold, a reprogramming recommendation for the implantable medical device including a third cardiac rhythm management therapy based on the second patient response metric; and
remotely reprogramming the implantable medical device to provide a third cardiac rhythm management therapy to the patient in a third time period subsequent to the second time period.

5. The system of claim 4, wherein determining the second patient response metric includes updating the first patient response metric.

6. The system of claim 4, wherein generating the reprogramming recommendation including the third cardiac rhythm management therapy comprises reverting from the second cardiac rhythm management therapy to the first cardiac rhythm management therapy if the second patient response metric indicates a worse patient status than the first patient response metric.

7. The system of claim 1, wherein the operations further comprise:
generating, based on determining that the value of the first patient response metric exceeds the first threshold, an alert to a user or process, and providing an indication of the value of the first patient response metric to the user or process.

8. The system of claim 1, wherein the operations further comprise:
scheduling an in-clinic follow-up appointment or adjusting a follow-up schedule for the patient based on the first patient response metric.

9. The system of claim 1, wherein the first cardiac rhythm management therapy includes a first mode or a first set of therapy parameters and the second cardiac rhythm management therapy includes a second mode different than the first mode or a second set of therapy parameters different than the first set of therapy parameters.

10. The system of claim 9, wherein the first mode comprises a CRT therapy mode and the second mode comprise an MSP mode different than the CRT therapy mode.

11. A method for evaluating patient response to cardiac rhythm management therapy by an implantable medical device and for remotely reprogramming the implantable medical device, the method comprising:
receiving physiologic information of a patient from the implantable medical device, including physiologic information of the patient in a first time period responsive to the implantable medical device providing a first cardiac rhythm management therapy to the patient;
determining, using the physiologic information of the patient in the first time period, a first patient response metric indicative of patient response to the first cardiac rhythm management therapy;
generating, based on determining that a value of the first patient response metric exceeds a first threshold, a reprogramming recommendation for the implantable medical device including a second cardiac rhythm management therapy based on the first patient response metric; and
remotely reprogramming the implantable medical device to provide a second cardiac rhythm management therapy to the patient in a second time period subsequent to the first time period,
wherein the first time period includes a post-implant time period following implant of the implantable medical device in the patient.

12. The method of claim 11, comprising:
receiving an indication of a time of implant of the implantable medical device in the patient, wherein the post-implant time period comprises a pre-determined time period from the time of implant of the implantable medical device.

13. The method of claim 12, wherein the pre-determined time period includes a time period of 2 to 4 weeks after the time of implant of the implantable medical device or after a recovery period after the time of implant of the implantable medical device.

14. The method of claim 11, comprising:
receiving physiologic information of the patient from the implantable medical device, including physiologic information of the patient in the second time period responsive to the implantable medical device providing the second cardiac rhythm management therapy to the patient;
determining, using the physiologic information of the patient in the second time period, a second patient response metric indicative of patient response to the second cardiac rhythm management therapy;
generating, based on determining that a value of the second patient response metric exceeds a second threshold, a reprogramming recommendation for the implantable medical device including a third cardiac rhythm management therapy based on the second patient response metric; and
remotely reprogramming the implantable medical device to provide a third cardiac rhythm management therapy to the patient in a third time period subsequent to the second time period.

15. The method of claim 14, wherein determining the second patient response metric includes updating the first patient response metric.

16. The method of claim 14, wherein generating the reprogramming recommendation including the third cardiac rhythm management therapy comprises reverting from the second cardiac rhythm management therapy to the first cardiac rhythm management therapy if the second patient response metric indicates a worse patient status than the first patient response metric.

17. The method of claim 11, comprising:
generating, based on determining that the value of the first patient response metric exceeds the first threshold, an alert to a user or process, and providing an indication of the value of the first patient response metric to the user or process.

18. The method of claim 11, comprising:
scheduling an in-clinic follow-up appointment or adjusting a follow-up schedule for the patient based on the first patient response metric.

19. The method of claim 11, wherein the first cardiac rhythm management therapy includes a first mode or a first set of therapy parameters and the second cardiac rhythm management therapy includes a second mode different than the first mode or a second set of therapy parameters different than the first set of therapy parameters.

20. The method of claim 19, wherein the first mode comprises a CRT therapy mode and the second mode comprise an MSP mode different than the CRT therapy mode.
